(19) United States
(12) Patent Application Publication
(10) Pub. No.: US 2026/0097053 A1
(43) Pub. Date: Apr. 9, 2026

(54) COMPOSITIONS AND METHODS FOR MODULATING CHOLESTEROL EFFLUX

(71) Applicant: CLEVELAND STATE UNIVERSITY, CLEVELAND, OH (US)

(72) Inventor: KAILASH GULSHAN, LEWIS CENTER, OH (US)

(21) Appl. No.: 19/184,214

(22) Filed: Apr. 21, 2025

Related U.S. Application Data
(60) Provisional application No. 63/636,390, filed on Apr. 19, 2024.

(51) Int. Cl. A61K 31/683; A61K 38/45; A61K 48/00; C12N 15/86

(52) U.S. Cl. CPC A61K 31/683; A61K 38/45; A61K 48/005; C12N 15/86; C12N 2750/14143; C12Y 207/01068

(57) ABSTRACT

Compositions and methods for diagnosing, treating, preventing, inhibiting, or ameliorating the symptoms of a disease or disorder that is modulated or otherwise affected by ABCA1 levels and cholesterol efflux or in which ABCA1 levels and cholesterol efflux is implicated are disclosed herein. PIP2 is shown to affect ABCA1 levels and promote RCT leading to reductions in unwanted cholesterol levels and atherosclerosis.

### CROSS-REFERENCE TO RELATED APPLICATIONS

This application claims priority to and any benefit of U.S. Provisional Application No. 63/636,390, filed Apr. 19, 2024, the content of which is incorporated herein by reference in its entirety.

### GOVERNMENT LICENSE RIGHTS

This invention was made with government support from the NHLBI grant no. R01-15848. The government has certain rights in the invention.

### SEQUENCE LISTING

The instant application contains a Sequence Listing which has been filed electronically in XML format and is hereby incorporated by reference in its entirety. Said XML copy, created on Aug. 7, 2025, is named 27433-04132 and is 13,766 bytes in size.

### TECHNICAL FIELD

The present disclosure is in the field of compositions and methods of providing one or more therapeutic interventions. More particularly, the present disclosure provides compositions and methods for diagnosing and treating cardiovascular disease and atherosclerosis.

### BACKGROUND

Cardiovascular disease is a leading cause of mortality and morbidity in the Under States and the broader developing world. Much of this has been attributed to western diets that increase blood cholesterol levels. Excess cholesterol can cause the formation of foam cells in the arterial intima, leading to atherosclerosis and cardiovascular disease (CVD). ABCA1 plays an atheroprotective role by removing cholesterol from peripheral tissue via the reverse cholesterol transport (RCT) pathway.

Lipid perturbations are the underlying cause of CVD. Cholesterol homeostasis is tightly regulated by the nuclear liver X receptor-Retinoid X receptor (LXR-RXR) heterodimer complex, which modulates the ATP-binding cassette protein ABCA1. Excess plasma cholesterol, especially in the form of oxidized low-density lipoprotein (LDL), promotes inflammation and atherosclerotic plaque growth. ABCA1 acts as an anti-atherosclerosis protein by removing excess cholesterol from arterial foam cells via the RCT pathway, and by dampening Toll-like receptor (TLR) signaling and inflammation.

### SUMMARY

The following is a brief summary of subject matter that is described in greater detail herein. This summary is not intended to be limiting as to the general inventive concepts or the scope of the claims.

Disclosed herein are various technologies pertaining to treatment for cardiovascular issues, including atherosclerosis and high cholesterol. In certain embodiments, the general inventive concepts contemplate compositions and methods for modifying/reducing blood lipid concentration. In certain embodiments, the general inventive concepts contemplate compositions and methods for modulating expression of one or more cholesterol efflux pathways. This, in turn leads to a modification/reduction in cholesterol in an individual.

There is a significant need for technological advancements to enhance diagnosis and treatment of CVD, high cholesterol, and atherosclerosis. The general inventive concepts are based, in part, on the discovery that cholesterol efflux transporter ABCA1 can be induced, leading to an increase in the reverse cholesterol transport pathway. Such inducement leads to a reduction in cholesterol and attendant benefits for patients with CVD.

In certain embodiments, the general inventive concepts contemplate a method of inducing expression of the cholesterol efflux transporter ABCA1.

The general inventive concepts provide a method to induce expression of cholesterol efflux transporter ABCA1. Increasing the activity of the RCT pathway (transport of excess cholesterol from peripheral tissues to the liver for excretion out of the body via ApoA1-ABCA1) is an established target for reducing atherosclerosis, but there are a limited number of methods available to increase activity of the RCT. One way proposed by scientists in the field is to use infusion of Apolipoprotein A1 (ApoA1). However, ApoA1 is a large protein. The general inventive concepts provide compositions and methods for inducing ABCA1 expression and unlike apoA1 infusion, no large protein preps are required. There is a significant unmet need for technological advancements to enhance CVD treatment and associated heart-related outcomes.

The general inventive concepts are based, in part, on the discovery that cholesterol loading increases nuclear PIP2, allowing binding of PIP2 to RXR via a novel PIP2 binding domain. PIP2 activates the LXR-RXR complex in conjunction with cholesterol derivatives, promoting the induction of ABCA1 and cholesterol efflux. Correspondingly, in low cholesterol conditions, nuclear PIP2 levels go down, rendering LXR-RXR deactivated, leading to reduced ABCA1 levels and dampened cholesterol efflux. See FIG. 1.

Described herein is a method of treating, preventing, inhibiting, or ameliorating the symptoms of a disease or disorder that is modulated or otherwise affected by ABCA1 levels and cholesterol efflux or in which ABCA1 levels and cholesterol efflux is implicated, comprising administering to an individual in need thereof a therapeutically effective amount of PIP2 or an expression vector capable of inducing PIP2 in the individual. In certain embodiments, the PIP2 is provided in a delivery vehicle, including but not limited to a vesicle such as a liposome. In certain embodiments, the expression vector capable of inducing PIP2 is rAAV8-PIP5K1a or a sequence having 80% to 99%. In certain embodiments, the sequence identity is 80%, 85%, 86%, 87%, 88%, 89%, 90%, 91%, 92%, 93%, 94%, 95%, 96%, 97%, 98%, 99% or 100%.

Also described is a method wherein the disease or disorder is hypercholesterolemia, hyperlipoproteinemia, hypertriglyceridemia, lipodystrophy, hyperglycemia, diabetes mellitus, dyslipidemia, atherosclerosis, Parkinson's disease, Alzheimer's disease, inflammation, immunological disorders, lipid disorders, obesity, efferocytosis, or cardiovascular disorders.

The general inventive concepts contemplate a method of treating dyslipidemia in an individual in need thereof. The method comprises providing a therapeutically effective amount of PIP2 or an expression vector capable of inducing PIP2 to an individual in need thereof, wherein the administration leads to a reduction in at least one of C-reactive protein and blood cholesterol concentration of the individual.

The general inventive concepts contemplate a method of reducing cholesterol-induced atherosclerosis in an individual in need thereof. The method comprises providing a therapeutically effective amount of PIP2 or an expression vector capable of inducing PIP2 to an individual in need thereof, wherein the administration leads to a reduction in blood cholesterol concentration of the individual.

The general inventive concepts contemplate a method of reducing the risk of cholesterol related cardiac events. The method comprises providing a therapeutically effective amount of PIP2 or an expression vector capable of inducing PIP2 to an individual in need thereof, wherein the administration leads to a reduction in blood cholesterol concentration of the individual.

The general inventive concepts contemplate a method of increasing cholesterol efflux from cells of a subject, comprising administering an effective cholesterol efflux-increasing amount of PIP2 or an expression vector capable of inducing PIP2 to an individual in need thereof, wherein the administration increases a level of cholesterol efflux.

The general inventive concepts contemplate a method of increasing expression of ATP-Binding Cassette (ABCA1) from cells of a subject, comprising administering an effective ABCA1 expression-increasing amount of PIP2 or an expression vector capable of inducing PIP2 to an individual in need thereof, wherein the administration increases a level of ABCA1 expression.

Other aspects and features of the general inventive concepts will become more readily apparent to those of ordinary skill in the art upon review of the following description of various exemplary embodiments in conjunction with the accompanying figures.

### BRIEF DESCRIPTION OF THE DRAWINGS

These and other features of the present disclosure will become better understood with regard to the following description and accompanying drawings in which:

FIG. 1 shows a model of cholesterol homeostasis.

FIG. 2A shows PIP2 levels in RAW cells and BMDMs ±50 μg/ml CD-Chol for 24 h.

FIG. 2B shows PIP2 levels ±2 mM cyclodextrin. N=5, mean±SD, *p<0.05, **P<0.01, ***P<0.005, by t test.

FIG. 2C is an image of confocal microscopy using PIP2 antibody showing PIP2 in ER-PM junctions (yellow arrows) and in nucleus in cholesterol-loaded cells.

FIG. 2D shows nuclear PIP2 levels in cholesterol loaded or depleted cells. N=5, mean±SD, *p<0.05, **P<0.01, ***P<0.005, by t test.

FIG. 3A shows ISA2011b reduced cellular PIP2 in THP-1 cells in dose-dependent fashion.

Figure B shows that ABCA1 expression was induced by T-compound (LXR agonist) and cholesterol efflux to apoA1 and NaTC was determined ±PIP2 depletion.

FIG. 3C is a Western blot of ABCA1 in THP-1 macrophages (PMA treated) ±T-compound ±PIP2 depletion.

FIG. 3D shows ABCA1 mRNA levels by qRT-PCR in THP-1 cells ±PIP2 depletion. N=4, mean±SD, *p<0.05, ***P<0.005, by t test).

FIG. 4 shows sequence alignment of RXR PIP2 binding domain with known PIP2 binding proteins. (SEQ ID NO.: 1-7)

FIG. 5A shows PONDER prediction for RXR, blue box represents PIP2 binding motif.

FIG. 5B is a helical wheel diagram of putative PIP2 domain of RXR.

FIG. 5C shows expression of native and Alexa-488 labeled WT-RXR.

FIG. 5D shows dipole potential showing PIP2 and RXR binding.

FIG. 5E shows zeta potential showing PIP2 and RXR binding.

FIG. 6A shows Full length (FL) Bodipy-TMR-PIP2 trafficking starting from plasma membrane (PM).

FIG. 6B shows Quantification of PIP2 intensity on PM over time.

FIG. 6C shows trafficking of short-chain PIP2 from PM to intracellular membranes.

FIG. 7 shows qRT-PCR of ABCA1. ABCA1 mRNA levels by qRT-PCR, normalized to beta actin. T-09 is LXR agonist used to induce ABCA1 expression.

FIG. 8A shows MST assay showing raw fluorescence traces of RXR with PIP2 (upper panel) and control liposomes (lower panel).

FIG. 8B shows RXR and PIP2 affinity by MST.

FIG. 8C shows a high-resolution STED microscopy showing RXR and PIP2 binding in GUVs.

FIG. 9 shows rAAV8 expression in mouse liver. Liver section of rAAV8 injected mouse showing GFP signal vs. mouse injected with vehicle.

FIG. 10A shows effect of rAAV8-PIP5K1a-3X-flag expression on ABCA1 levels in mice. WT C57BL6 mice were injected with rAAV8-PIP5K1a or an empty viral vector. 5-weeks post-transduction, mice were sacrificed, and tissue harvested. Liver homogenates were probed with antibodies against Flag tag, ABCA1, LDLr, and beta-actin.

FIG. 10B is an image showing intestinal tissue was separated to isolate the Ileum. Protein extracts of Ileum were probed with antibodies against ABCA1 and beta-actin.

FIG. 10C is a bar graph showing qRT-PCR analysis of ABCA1 mRNA from injected mice's liver, beta-actin mRNA was used as control.

FIG. 10D is a bar graph showing qRT-PCR analysis of ABCA1 mRNA from injected Ileum tissue, beta-actin mRNA was used as control.

FIG. 11A is a bar graph showing the effect of rAAV8-PIP5K1a-3X-flag expression on reverse cholesterol transport (RCT) in mice plasma at 24 h, 48 h, and 72 h.

FIG. 11B is a bar graph showing the effect of rAAV8-PIP5K1a-3X-flag expression on reverse cholesterol transport (RCT) in mice liver at 72 h.

FIG. 12 is a vector map for an AAV vector according to the general inventive concepts (i.e., rAAV8-PIP5K1a).

FIG. 13 shows a position explanation for an AAV vector according to the general inventive concepts.

FIG. 14 is a sequence for an AAV vector according to the general inventive concepts (rAAV8-PIP5K1a). (SEQ ID NO.: 8)

### DETAILED DESCRIPTION

Various technologies pertaining to a composition, system, and method for treating CVD, high cholesterol, atherosclerosis, and other cardiovascular related conditions are described herein. The general inventive concepts provide a method to induce expression of cholesterol efflux transporter ABCA1 to treat one or more conditions or diseases described herein.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

As used herein, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

Any combination of method or process steps as used herein may be performed in any order, unless otherwise specified or clearly implied to the contrary by the context in which the referenced combination is made.

The terms “susceptible” and “at risk” as used herein, unless otherwise specified, mean having little resistance to a certain condition or disease relative to the general population, including being genetically predisposed, having a family history of, and/or having symptoms of the condition or disease. The term refers to those having a vulnerability higher than the general population.

The terms “modulating” or “modulation” or “modulate” as used herein, unless otherwise specified, refer to the targeted movement of a selected characteristic. In certain embodiments, the characteristic may be cholesterol levels, including HDL and/or LDL levels, reduction in arterial plaques, angiography, ultrasound, reduction in calcification (e.g., by CT scan).

The term “ameliorate” as used herein, unless otherwise specified, means to eliminate, delay, or reduce the prevalence or severity of symptoms associated with a condition.

The terms “an effective amount” and “a therapeutically effective amount” are used interchangeably herein and are intended to qualify the amount of a composition according to the general inventive concepts which will achieve the goal of decreasing the risk that the individual will suffer an adverse health event (e.g., cardiovascular event related to high cholesterol), including reducing one or more symptoms, while avoiding adverse side effects such as those typically associated with alternative therapies. The effective amount may be administered in one or more doses.

The terms “treating”, and “treatment” as used herein, unless otherwise specified, includes delaying the onset of a condition, reducing the severity of symptoms of a condition, or eliminating some or all of the symptoms of a condition.

In specific embodiments of the invention, the methods and compositions are provided to an individual, where the individual may be a human adult or geriatric subject.

The general inventive concepts provide a method to induce expression of cholesterol efflux transporter ABCA1. Increasing the activity of the RCT pathway (transport of excess cholesterol from peripheral tissues to the liver for excretion out of the body via ApoA1-ABCA1) is an established target for reducing atherosclerosis, but there are a limited number of methods available to increase activity of the RCT. One way proposed by scientists in the field is to use infusion of Apolipoprotein A1 (ApoA1). However, ApoA1 is a large protein. The general inventive concepts provide compositions and methods for inducing ABCA1 expression and unlike apoA1 infusion, no large protein preps are required. There is a significant unmet need for technological advancements to enhance CVD treatment and associated heart-related outcomes.

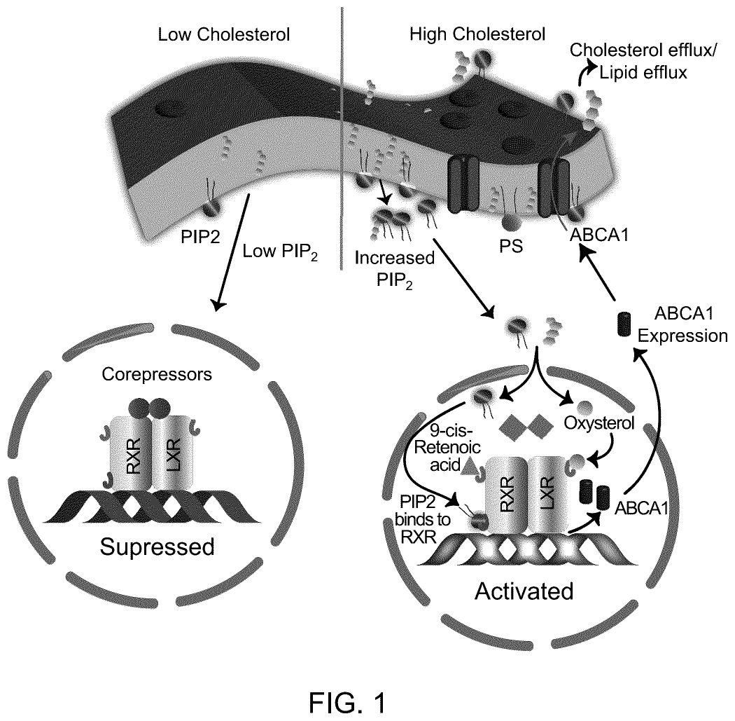

The general inventive concepts are based, in part, on the discovery that cholesterol loading increases nuclear PIP2, allowing binding of PIP2 to RXR via a novel PIP2 binding domain. PIP2 activates the LXR-RXR complex in conjunction with cholesterol derivatives, promoting the induction of ABCA1 and cholesterol efflux. Correspondingly, in low cholesterol conditions, nuclear PIP2 levels go down, rendering LXR-RXR deactivated, leading to reduced ABCA1 levels and dampened cholesterol efflux. See FIG. 1.

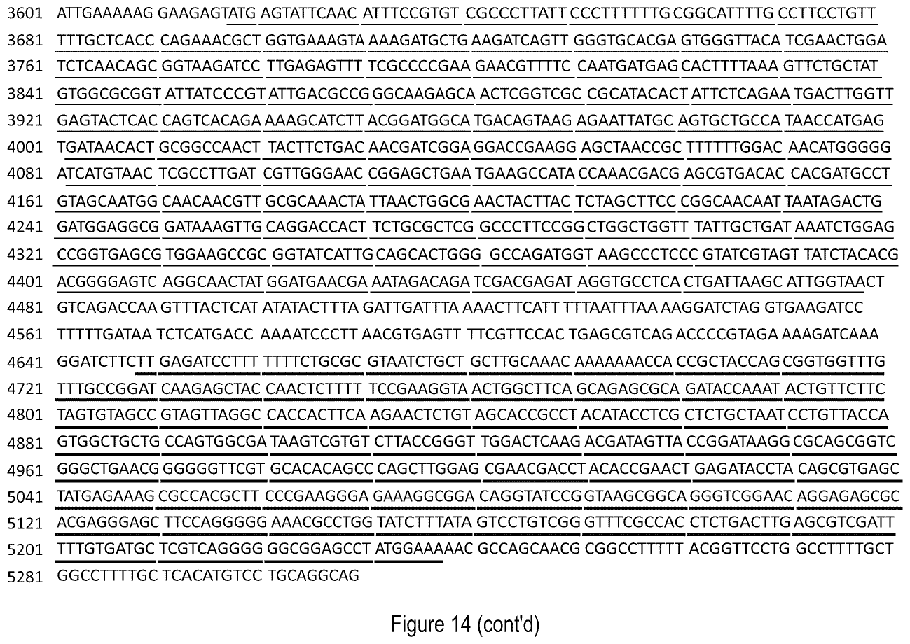

Described herein is a method of treating, preventing, inhibiting, or ameliorating the symptoms of a disease or disorder that is modulated or otherwise affected by ABCA1 levels and cholesterol efflux or in which ABCA1 levels and cholesterol efflux is implicated, comprising administering to a subject in need thereof a therapeutically effective amount of PIP2 or an expression vector capable of inducing PIP2 in the individual. In certain embodiments, the expression vector capable of inducing PIP2 is rAAV8-PIP5Ka (see FIG. 14) or a sequence having 80% to 99%. In certain embodiments, the sequence identity is 80%, 85%, 86%, 87%, 88%, 89%, 90%, 91%, 92%, 93%, 94%, 95%, 96%, 97%, 98%, 99% or 100%.

Also described is a method wherein the disease or disorder is hypercholesterolemia, hyperlipoproteinemia, hypertriglyceridemia, lipodystrophy, hyperglycemia, diabetes mellitus, dyslipidemia, atherosclerosis, Parkinson's disease, Alzheimer's disease, inflammation, immunological disorders, lipid disorders, obesity, efferocytosis, or cardiovascular disorders.

The general inventive concepts contemplate a method of treating dyslipidemia in an individual in need thereof. The method comprises providing a therapeutically effective amount of PIP2 or an expression vector capable of inducing PIP2 to an individual in need thereof, wherein the administration leads to a reduction in blood cholesterol concentration of the individual.

The general inventive concepts contemplate a method of treating inflammation in an individual in need thereof. The method comprises providing a therapeutically effective amount of PIP2 or an expression vector capable of inducing PIP2 to an individual in need thereof, wherein the administration leads to a reduction in plasma C-reactive protein levels of the individual.

The general inventive concepts contemplate a method of reducing cholesterol-induced atherosclerosis in an individual in need thereof. The method comprises providing a therapeutically effective amount of PIP2 or an expression vector capable of inducing PIP2 to an individual in need thereof, wherein the administration leads to a reduction in blood cholesterol concentration (e.g., HDL/LDL levels) of the individual.

The general inventive concepts contemplate a method of reducing the risk of cholesterol related cardiac events. The method comprises providing a therapeutically effective amount of PIP2 or an expression vector capable of inducing PIP2 to an individual in need thereof, wherein the administration leads to a reduction in blood cholesterol concentration of the individual.

The general inventive concepts contemplate a method of increasing cholesterol efflux from cells of a subject, comprising administering an effective cholesterol efflux-increasing amount of PIP2 or an expression vector capable of inducing PIP2 to an individual in need thereof, wherein the administration increases a level of cholesterol efflux.

The general inventive concepts contemplate a method of increasing expression of ATP-Binding Cassette (ABCA1) from cells of a subject, comprising administering an effective ABCA1 expression-increasing amount of PIP2 or an expression vector capable of inducing PIP2 to an individual in need thereof, wherein the administration increases a level of ABCA1 expression.

In accordance with the methods of the present invention, PIP2 or an expression vector configured to increase expression thereof, may be administered to the individual in need thereof for a time period of at least 2 days, or at least 3 days, or at least 5 days, or at least 6 days, or at least 1 week, or at least 2 weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 14 weeks, or at least 16 weeks, or at least 18 weeks, or at least 24 weeks or longer. In specific embodiments of the methods, PIP2 or an expression vector configured to increase expression thereof, are administered to an individual once or multiple times daily or weekly. In specific embodiments, the, PIP2 or an expression vector configured to increase expression thereof, are administered to the subject from about 1 to about 6 times per day or per week, or from about 1 to about 5 times per day or per week, or from about 1 to about 4 times per day or per week, or from about 1 to about 3 times per day or per week. In other specific embodiments, the PIP2 or an expression vector configured to increase expression thereof, are administered once or twice daily for a period of at least 2 days, at least 3 days, at least 4 days, at least 5 days or at least 6 days, or at least one week, at least two weeks, at least three weeks, at least four weeks, at least five weeks, or at least six weeks.

In certain embodiments of the methods of the invention, the individual in need thereof is administered a therapeutically effective amount of PIP2, including administration via liposome vehicle. This amount will vary depending on the individual and the specific therapeutic endpoint. In certain embodiments of the methods of the invention, the individual in need thereof is administered from about 50-400 mg/kg to about 150-200 mg/kg grams per day of PIP2. In further embodiments of the methods of the invention, the individual is administered from about 50 mg/kg to about 350 mg/kg, including about 50 mg/kg to about 300 mg/kg, including about 50 mg/kg to about 275 mg/kg, including about 50 mg/kg to about 265 mg/kg, including about 50 mg/kg to about 250 mg/kg, including about 50 mg/kg to about 235 mg/kg, including about 50 mg/kg to about 220 mg/kg, including about 50 mg/kg to about 210 mg/kg. In further embodiments of the methods of the invention, the individual is administered from about 70 mg/kg to about 400 mg/kg, including from about 85 mg/kg to about 400 mg/kg, including from about 100 mg/kg to about 400 mg/kg, including from about 115 mg/kg to about 400 mg/kg, including from about 125 mg/kg to about 400 mg/kg, including from about 145 mg/kg to about 400 mg/kg, including from about 165 mg/kg to about 400 mg/kg, including from about 200 mg/kg to about 400 mg/kg, including from about 240 mg/kg to about 400 mg/kg, including from about 285 mg/kg to about 400 mg/kg, and including from about 325 mg/kg to about 400 mg/kg. In certain embodiments, PIP2 is provided with long circulating and pH-sensitive (PEG2000-DSPE/DOPC/CHOL/PIP2. In certain embodiments, the general composition of liposomes according to embodiments of the general inventive concepts is as follows; DOPC lipid (180 mg), DOPE lipid (60 mg), Cholesterol (8 mg), PEG-DSPE lipid (60 mg), PIP2 lipid (20.8 mg). These lipids are typically dissolved in 15 ml solution.

In certain embodiments, the general inventive concepts contemplate compositions and methods for reducing blood lipid concentration. In certain embodiments, the general inventive concepts contemplate compositions and methods for modulating expression of one or more cholesterol efflux pathways. This, in turn leads to a reduction in cholesterol in an individual.

Lipid perturbations are the underlying cause of CVD. Cholesterol homeostasis is tightly regulated by the nuclear liver X receptor-Retinoid X receptor (LXR-RXR) heterodimer complex, which modulates the ATP-binding cassette protein ABCA1. Excess plasma cholesterol, especially in the form of oxidized low-density lipoprotein (LDL), promotes inflammation and atherosclerotic plaque growth. ABCA1 acts as an anti-atherosclerosis protein by removing excess cholesterol from arterial foam cells via the RCT pathway, and by dampening Toll-like receptor (TLR) signaling and inflammation.

PIP2 plays a role in the ABCA1-mediated cholesterol efflux pathway, but up until now, it was not clear if PIP2 plays a direct role in regulating cellular cholesterol homeostasis. The general inventive concepts are based on the recognition that cholesterol loading led to PIP2 trafficking to ER-PM junctions and increased nuclear PIP2 levels. Reduction in cellular PIP2 led to reduced ABCA1 levels, while an increase in PIP2 levels led to increased ABCA1 levels. PIP2 interacts with the LXR-RXR complex by directly binding to RXR via a novel PIP2 binding domain in RXR. The data shown herein demonstrate that cellular PIP2 levels are directly related to cholesterol load, and PIP2 modulates cholesterol efflux pathways to maintain appropriate cholesterol levels.

In view of these findings, Applicants have shown that a new model of lipid homeostasis should be considered, where cholesterol regulates PIP2 levels/localization, and PIP2 in-turn regulates ABCA1 expression via RXR. Based on Applicant's findings, it was determined that PIP2 may serve as a sensor of cholesterol pool, relaying the status of cholesterol levels to LXR/RXR complex, allowing appropriate modulation of ABCA1 to maintain cholesterol homeostasis.

Cholesterol derivatives, such as oxysterols, can activate LXR, but it's not fully clear if, and how, PIP2 plays a role in the cholesterol-mediated activation of the LXR-RXR complex. Applicant has shown that cholesterol loading increases PIP2, and PIP2 in turn increase ABCA1 expression via activating RXR-LXR transcription factor complex. Applicant has also shown that administration of an expression vector that promotes the expression of PIP2 can increase RCT in an individual.

In the presence of excess cholesterol LXR-RXR is activated, leading to induction of genes involved in cholesterol efflux, such as ABCA1, and suppression of genes involved in cholesterol uptake, such as LDL receptor (LDLr). Applicant has shown that cholesterol loading led to PIP2 trafficking to ER-PM junctions and increased nuclear PIP2 levels. Interestingly, reduction in cellular PIP2 led to reduced ABCA1 levels, while elevation in cellular PIP2 increased ABCA1 levels. PIP2 interacts with the LXR-RXR complex by directly binding to RXR. These data show that cellular PIP2 levels are directly related to cholesterol load, and PIP2 modulates ABCA1 levels to maintain appropriate cholesterol levels. This data points toward a new model of lipid homeostasis, where cholesterol regulates PIP2 levels and localization, and PIP2 in-turn regulates ABCA1 expression via RXR.

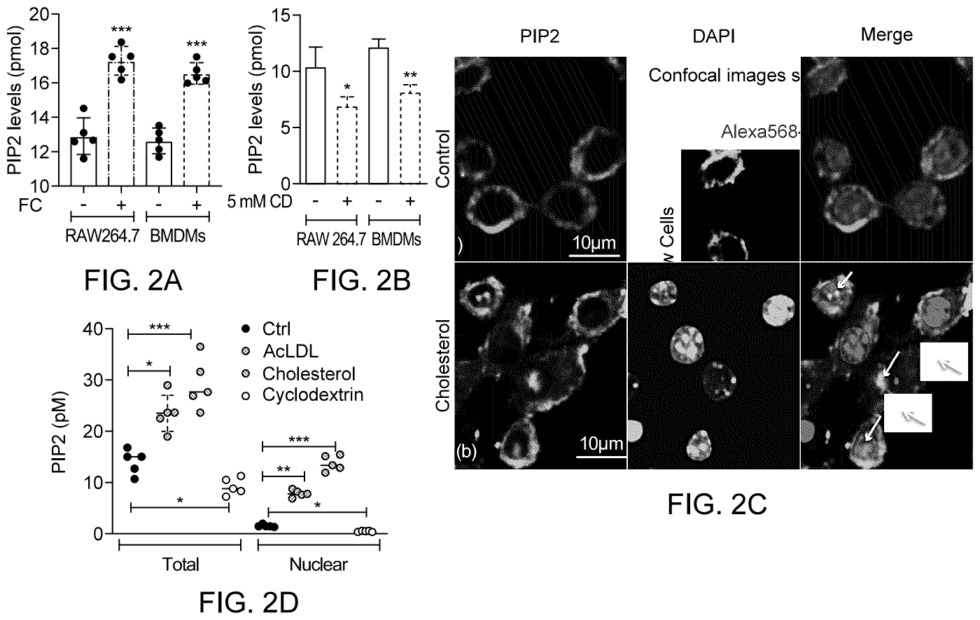

PIP2 regulates ABCA1 expression. The levels of cholesterol efflux pump ABCA1 are known to respond to cellular cholesterol pools. As PIP2 levels were modulated by cholesterol, Applicant sought to determine if PIP2 can regulate ABCA1 levels. Treatment of THP-1 macrophages with ISA2011b (a potent inhibitor of PIP2 biosynthetic enzyme PIP5Kα), or with siRNA against PIP5Kα markedly reduced cellular PIP2 levels, reduced cholesterol efflux to acceptors such as apoA1 and bile salt sodium taurocholate (NaTC), and reduced ABCA1 levels (FIG. 2A, 2B, 2C). Since cholesterol loading increases nuclear PIP2, Applicants believe that PIP2 may be affecting either transcription or stability of ABCA1 mRNA transcript. Pilot data showed reduction in ABCA1 mRNA in THP-1 cells treated with ISA2011b or PIP5Kα siRNA. (FIG. 2D), supporting the hypothesis that PIP2 may be regulating levels of ABCA1 mRNA.

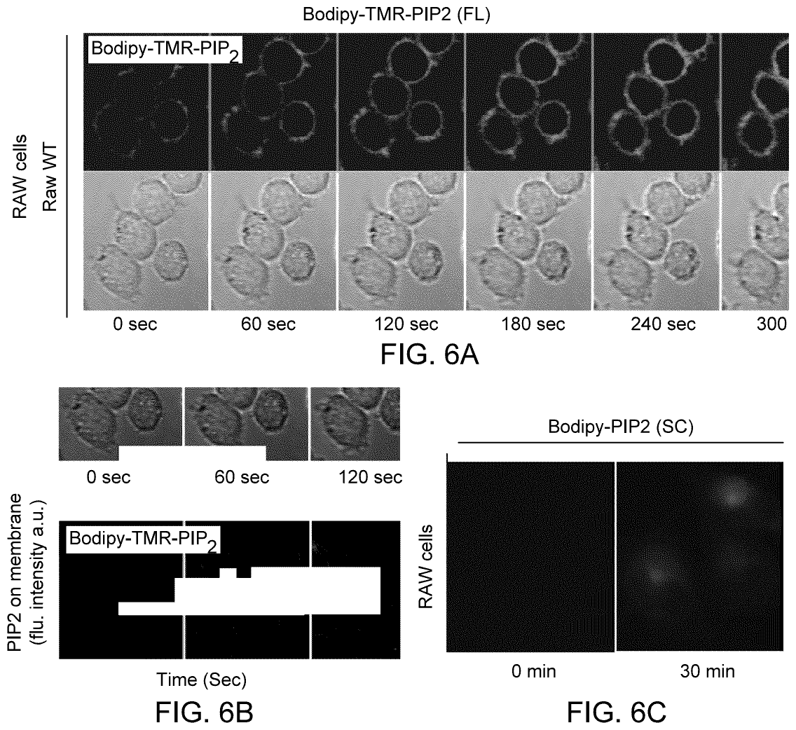

RXR contains a PIP2 binding domain. The bona fide PIP2 binding proteins contain pleckstrin homology (PH) domain. In addition to PH domain, the PIP2 binding motif KXXXXXXXXK/RXR is present in many well-known PIP2 binding proteins. Using computational analysis, Applicant identified a putative PIP2 binding motif in RXR with a sequence KDCLIDKRQRNR (SEQ ID NO.: 7) (FIG. 5). The PIP2 binding domain is located between DNA binding domain and retinoic acid binding domain of RXR and is conserved among various species.

There is a significant need for technological advancements to enhance diagnosis and treatment of CVD, high cholesterol, and atherosclerosis. The general inventive concepts are based, in part, on the discovery that cholesterol efflux transporter ABCA1 can be induced, leading to an increase in the reverse cholesterol transport pathway. Such inducement leads to a reduction in cholesterol and attendant benefits for patients with CVD.

In certain embodiments, the general inventive concepts contemplate a method of inducing expression of the cholesterol efflux transporter ABCA1.

The following examples illustrate features and/or advantages of the compositions, systems, and methods according to the general inventive concepts. The examples are given solely for the purpose of illustration and are not to be construed as limitations of the general inventive concepts, as many variations thereof are possible without departing from the spirit and scope of the general inventive concepts.

### EXAMPLES

As mentioned, lipid perturbations are the underlying cause of atherosclerosis, cardiovascular disease (CVD), and other metabolic diseases. Excess plasma cholesterol, especially in the form of oxidized low-density lipoprotein (LDL), promotes plaque growth and inflammation in atherosclerotic lesions. ABCA1 acts as an anti-atherosclerosis protein by removing excess cholesterol from arterial foam cells via reverse cholesterol transport (RCT) pathway, and by dampening Toll-like receptor (TLR) signaling and inflammation.

In a previous AHA-funded SDG project, ABCA1 was identified as the first known phosphatidylinositol 4, 5-bisphosphate (PIP2) floppase, and this finding was later confirmed by other studies.

PIP2 flop (translocation of PIP2 from the inner leaflet of plasma membrane to the cell surface) promoted cholesterol efflux from cells, indicating cross-talk between cholesterol and PIP2 in maintaining lipid homeostasis. The role and mechanism of PIP2 in regulating cholesterol homeostasis, RCT, and atherosclerosis are not clear.

Applicants made several new and exciting findings regarding PIP2-cholesterol interplay: 1) cholesterol loading leads to PIP2 trafficking to endoplasmic-plasma membrane (ER-PM) junctions and increased nuclear PIP2 levels, 2) expression of ABCA1 correlated with levels of cellular PIP2 and 3) PIP2 interacted with LXR-RXR complex in a cooperative fashion to regulate ABCA1 expression. Our preliminary data points toward a new model of lipid homeostasis, where cholesterol regulates PIP2 levels/localization, and PIP2 in turn regulates ABCA1 expression via RXR. Based on these data, we came up with an exciting hypothesis: “As cellular PIP2 levels are much lower than cholesterol, PIP2 may serve as an early sensor of cellular cholesterol pool, relaying the status of cholesterol levels to LXR/RXR complex, allowing appropriate modulation of ABCA1 to maintain cholesterol homeostasis (FIG. 1).

Determining the trafficking of PIP2 and PIP2 metabolic enzymes in cholesterol-loaded or depleted cells. Applicants will confirm that cholesterol increases cellular PIP2 and promotes redistribution of PIP2 to the nucleus. Using cholesterol-loaded and depleted conditions; we will determine trafficking of PIP2 from PM to nucleus via ER-PM junctions, and nuclear-cytoplasmic shuttling of the PIP2 metabolic enzyme.

Determining the role of PIP2 in CVD in mice. Applicants will confirm that PIP2 can promote RCT and reduce atherosclerosis. Various mouse models of RCT and atherosclerosis will be used to test a high-risk high-reward hypothesis that PIP2 supplementation can serve as an anti-CVD therapeutic.

While not wishing to be bound by any particular theory, Applicants hypothesize that the cells can discern an increase or decrease in PIP2 much earlier (due to much lower basal PIP2 levels vs. other lipids) than perceiving alterations in cholesterol levels, allowing dampening or ramping up of ABCA1 expression to fine tune cholesterol levels.

In comparison to other phospholipids, PIP2 has a high critical micelle concentration (>10 uM vs. 0.46 nM for 16:0 PC). Critical micelle concentration is defined as the lipid monomer concentration at which >5% of the total micellar aggregates begin to appear in equilibrium. Furthermore, certain fatty acid species of PIP2 can spontaneously desorb from the membrane bilayer into the aqueous phase over time. This property of PIP2 allows it to diffuse in an aqueous environment and may promote interaction with LXR-RXR to regulate cholesterol homeostasis.

Excess cholesterol can cause the formation of foam cells in the arterial intima, leading to atherosclerosis and CVD, while ABCA1 plays an atheroprotective role by removing cholesterol from peripheral tissue via the RCT pathway. Cholesterol derivatives, such as oxysterols, can activate LXR, but it's not fully clear if, and how, PIP2 plays a role in the cholesterol-mediated activation of the LXR-RXR complex. Our proposal challenges the existing paradigm in the field by establishing PIP2 as a novel player in LXR-RXR mediated induction of ABCA1. This project will provide mechanistic insights into the novel role of PIP2 in cholesterol homeostasis and also provide pre-clinical evidence for PIP2 as an RCT-promoting and anti-atherosclerotic therapeutics.

The general inventive concepts are based, at least in part, on the following discoveries:
Cholesterol-induced trafficking of PIP2 to endoplasmic reticulum-plasma membrane (ER-PM) junctions and increase in nuclear PIP2.
PIP2 mediated regulation of ABCA1 levels and cholesterol efflux.
Identification of novel PIP2 binding domain of RXR, and functional analysis of this domain.
Identification of novel regulatory pathway in which cholesterol increases PIP2, and PIP2 in turn induces ABCA1 expression via LXR-RXR, leading to enhanced cholesterol efflux,
Translational strategies for testing the direct role of PIP2 in RCT and atherosclerosis.
Cutting-edge techniques and tools: STED and STORM microscopy, rAAV8 expression, and a variety of cell-free systems.

Cholesterol loading increases PIP2 levels and induces nuclear localization of PIP2. Previous work has shown the role of PIP2 in ABCA1 mediated cholesterol efflux, and cholesterol-induced alteration in lipid species, such as sphingomyelin. Given the role of PIP2 in cholesterol efflux pathway, we sought to determine if PIP2 levels are altered by cholesterol. Loading of RAW264.7 macrophages or mouse BMDMs with free-cholesterol (cyclodextrin-cholesterol) leads to a significant increase in cellular PIP2 levels (FIG. 2A). Similar results were found with acetylated low-density lipoprotein (AcLDL) loading (not shown). In contrast, depletion of cellular cholesterol by cyclodextrin led to ˜35% reduction in cholesterol levels (not shown) and a significant decrease in cellular PIP2 levels (FIG. 2B).

Interestingly, cholesterol loading led to redistribution of PIP2 from plasma membrane to membrane structures resembling endoplasmic reticulum-plasma membrane (ER-PM) junctions (FIG. 2C, yellow arrows) and to nucleus (FIG. 2C, white arrows). To quantify nuclear PIP2, intact nuclei were separated from cells using nuclei floatation method and nuclear PIP2 levels were markedly higher in cholesterol loaded vs unloaded cells, and reduced in cholesterol depleted cells vs. control (FIG. 2D) The increase in PIP2 was specific, as there was no change in PI3P levels (not shown). These data indicated the intricate and specific relation of cholesterol with total and nuclear PIP2. The mechanism for cholesterol-induced increase in PIP2 is not clear. Applicants will confirm if the cholesterol-induced increase in nuclear PIP2 is due to altered trafficking of PIP2 metabolic enzymes or due to increased import of PIP2 into nucleus.

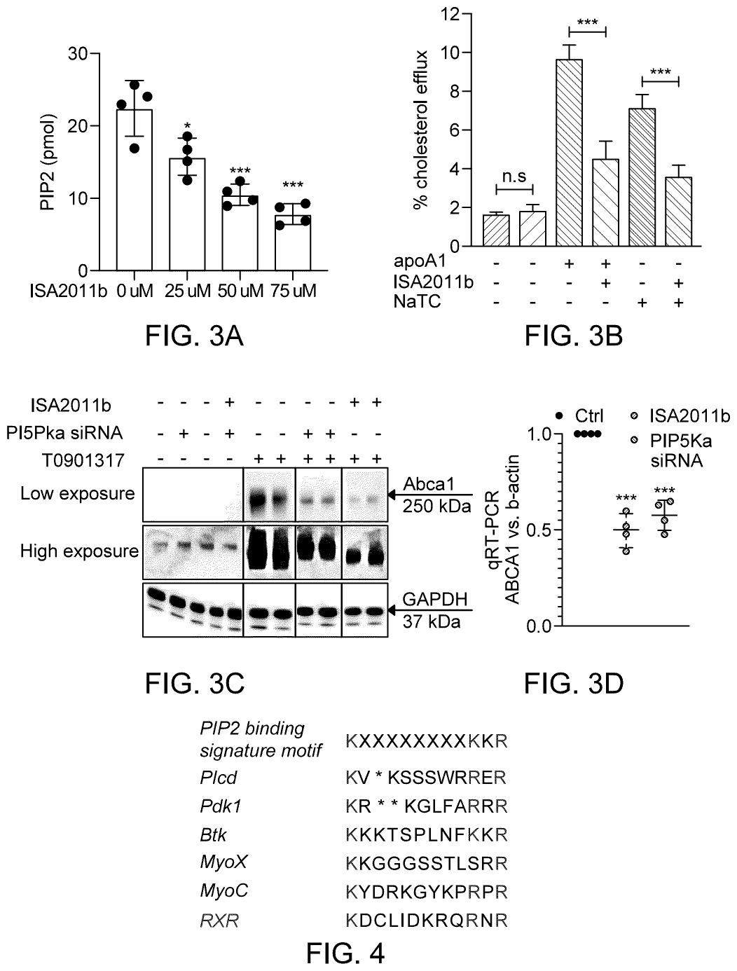

The levels of cholesterol efflux pump ABCA1 are known to respond to the levels of cellular cholesterol. As PIP2 levels were modulated by cholesterol, we sought to determine if PIP2 can regulate ABCA1 levels. Treatment of THP-1 macrophages with ISA2011b (a potent inhibitor of PIP2 biosynthetic enzyme PIP5Kα), or with siRNA against PIP5Kα markedly reduced cellular PIP2 levels, reduced cholesterol efflux to acceptors (such as apoA1 and bile salts), and reduced ABCA1 levels (FIG. 3A, 3B, 3C). Since cholesterol loading increases nuclear PIP2, our hypothesis is that PIP2 may be effecting either transcription or stability of ABCA1 transcript. Pilot data showed reduction in ABCA1 mRNA in THP-1 cells treated with ISA2011b or PIP5Kα depletion. (FIG. 3D), supporting our hypothesis that PIP2 may be regulating levels of ABCA1 mRNA. Applicants shown before that knockdown of PIP2 phosphatase TMEM55b that dephosphorylates PIP2 to PI5P) in macrophages increased PIP2 levels, and this finding was later confirmed in hepatocytes and mice.

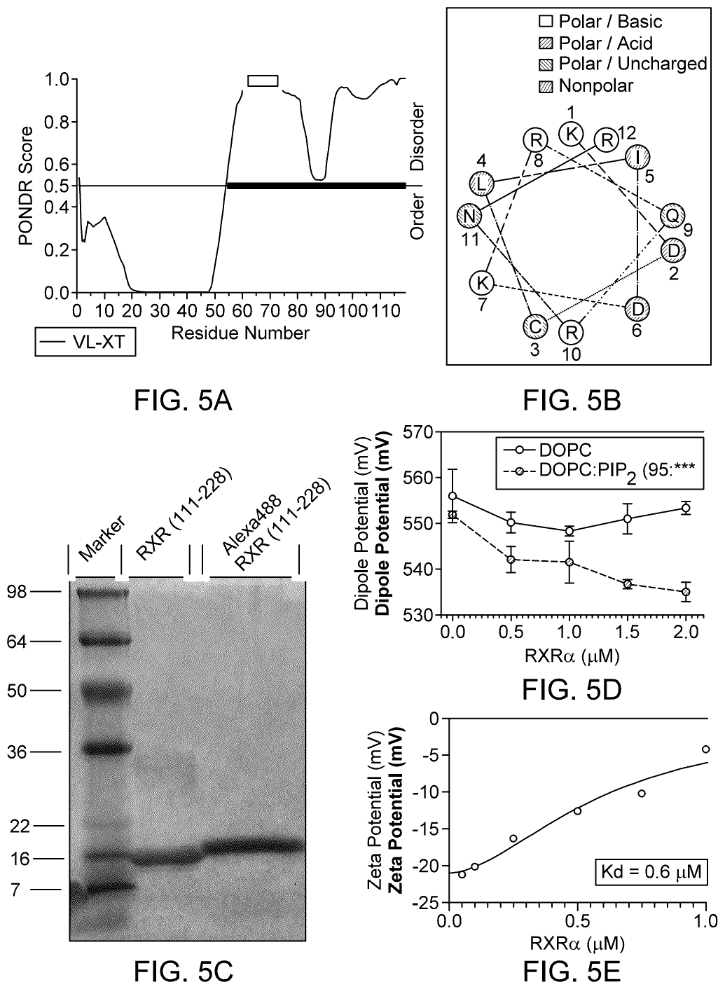

One of the way cells can alter nuclear PIP2 is by modulating levels of PIP2 metabolic enzymes in nucleus. Thus, Applicants probed the localization of endogenous TMEM55b in macrophages under normal growth condition (DMEM+10% FBS). The cytoplasmic and nuclear fractions were separated and blotted for TMEM55b along with controls (lamin for nuclear fraction and tubulin for cytoplasmic fraction). As shown in FIG. 4A, a small portion of TMEM55b is present in nucleus under basal conditions. Next, we used two independent strategies to increase cellular PIP2 levels and to determine if increasing PIP2 can increase ABCA1. In first strategy, we used TMEM55b KO in hepatocytes and mice. The siRNA mediated knockdown of PIP2 phosphatase TMEM55b in HePG2 cells significantly increased ABCA1 levels (FIG. 4B), and liver tissue from C57BL6J-TMEM55b^{-/-} KO mice also showed higher ABCA1 protein levels vs C57BL6J-WT mice (FIG. 4C). The tissue samples of TMEM55b KO mice were kindly provided by Medina lab. Thus, using several strategies of altering PIP2 levels, we show the in-vitro and in-vivo role of PIP2 in regulating cholesterol efflux and ABCA1 levels.

RXR contains a PIP2 binding domain. The bona fide PIP2 binding proteins contain pleckstrin homology (PH) domain. In addition to PH domain, the PIP2 binding motif KXXXXXXXXK/RXR is present in many well-known PIP2 binding protein. Using computational analysis, we identified a conserved putative PIP2 binding motif in RXR with a sequence KDCLIDKRQRNR (SEQ ID NO.: 7) (FIG. 5).

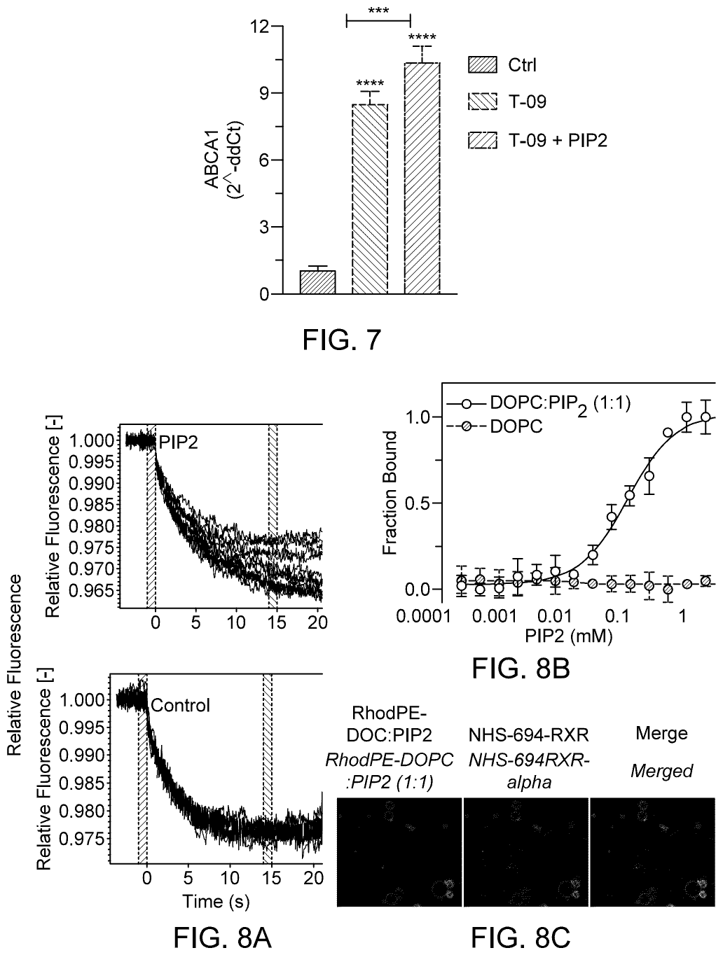

The PIP2 binding domain of RXR is conserved among various species. The PONDER prediction for RXR region surrounding PIP2 binding motif (112 amino acids with 50 amino acids on each side of PIP2 binding motif) showed that the region containing PIP2 binding motif is highly disordered as compared to surrounding region (FIG. 6A), indicating the interface for interaction with other molecules. A helical wheel diagram of putative PIP2 binding sequence (KDCLIDKRQRNR) (SEQ ID NO.: 7) shows a positively charged binding surface based on the alignment of polar residues such as K and R (FIG. 6B). To test binding of PIP2 with RXRα, we used WT-RXR protein (˜117 AA, 50 AA on N-terminal side of PIP2 binding domain and 55 AA on C-terminal side, with size of ˜13 KDa). We have purified untagged and Alexa-488 labeled RXR protein (FIG. 6C) and used these proteins for pilot studies.

PIP2 or control liposomes doped with Di 8-ANEPPS (Ex 420 nm, Em 520 nm) were incubated with increasing concentrations of RXRα protein and shift in spectra was used to detect interaction between protein and membrane. The ratio (R) of Fi 420/F1 520 was used to calculate dipole potential using the formula: Dipole potential (mV)=R+0.3/4.3×10³. Pilot data shows that the dipole potential of PIP2 liposomes decreased markedly in presence of RXR protein, indicating dose-dependent interaction, while the control liposomes showed no interaction with RXR (FIG. 6D).

To evaluate the affinity of RXR toward PIP2, 200 μM DOPC ±PIP2 (DOPC:PIP2 95:5 mole %) liposomes were incubated with increasing concentration of RXR protein and membrane potential was determined using Zetasizer Nano ZS (Malvern Zetasizer Nano ZS90, Netherland) with a 633 nm Laser. Surface potential of each sample was calculated using the Smoluchowsky equation. Pilot data show Zeta potential of membrane containing PIP2 is reduced in presence of RXR protein, while control liposomes showed no effect (FIG. 6E).

Research design and methods: Determining the trafficking of PIP2 and PIP2 metabolic enzymes in cholesterol loaded or depleted cells. Cholesterol loading increases nuclear PIP2 either via trafficking of PIP2 from plasma membrane to nucleus via ER-PM junctions or via alteration in nuclear localization of PIP2 biosynthetic or degradadtive enzymes.

Cholesterol loaded macrophages showed higher total and nuclear PIP2 levels and PIP2 was enriched in ER-PM junctions. Thus, PIP2 can be directly transported from PM to nucleus via ER-PM junctions. Thus, Applicants will use a fluorescent PIP2 tracker to determine trafficking of PIP2. Increased nuclear PIP2 in cholesterol-loaded cells could also be due to increased import or reduced export of PIP2 metabolic enzymes from nucleus. A small portion of PIP2 phosphatase TMEM55b (FIG. 4A) and PIP2 biosynthetic enzyme PIP5K 1a is present in the nucleus under basal conditions. Thus, we propose to test if localization of PIP2 metabolic enzymes is altered in cholesterol loaded cells.

To decipher the mechanism of cholesterol-induced increase in nuclear PIP2, we will use two cell types: Macrophages (mouse RAW264.7, BMDMs, and human THP-1), and hepatocytes (HepG2). For cholesterol loading, we will use AcLDL (100 μg/ml for 24 h) or cyclodextrin-cholesterol (50 μg/ml for 24 h). For cholesterol depletion, we will use lipoprotein deficient serum (LPDS) media+statin treatment (2 μM) for HepG2 and THP-1 cells and cyclodextrin for RAW264.7 cells (as statins are not effective in mouse cells).

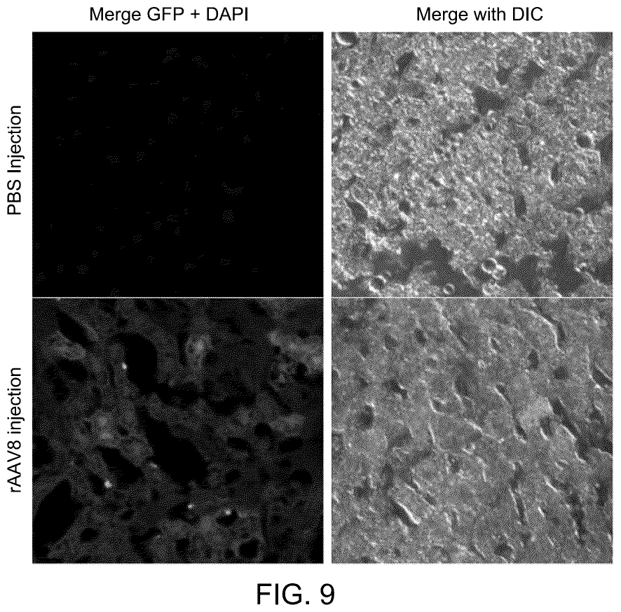

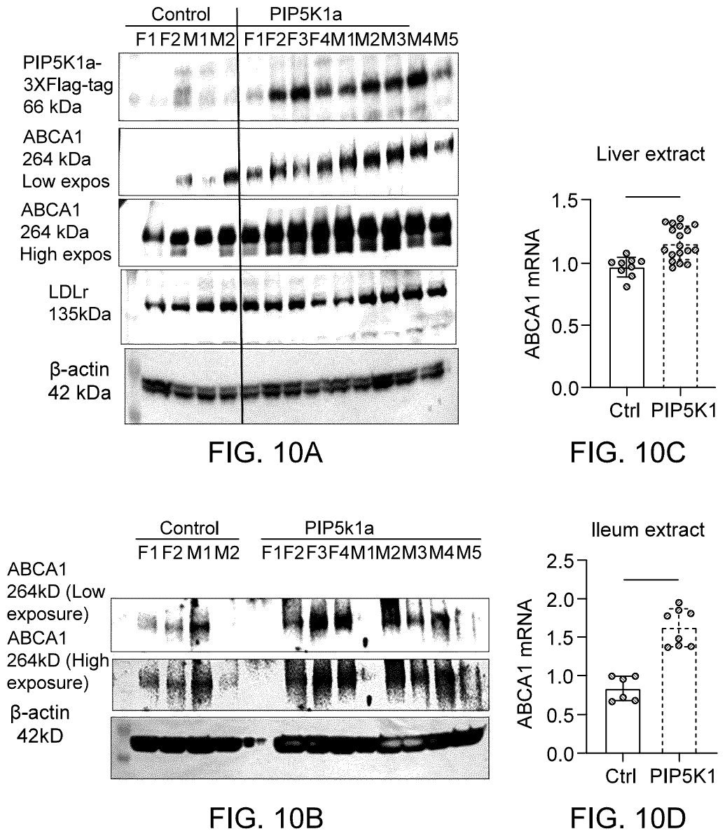

Levels and localization of TMEM55b or PIP5Kα will be compared in cholesterol depleted, cholesterol loaded, and control cells. To test if PIP2 is directly imported from cell membranes into nucleus, cells will be loaded with cholesterol along with Bodipy-TMR-PIP2 (full length and short chain), followed by confocal microscopy. We have established assays to study trafficking of PIP2 using variety of labels, supporting the feasibility of proposed studies. FIGS. 7A and 7B shows the time-course and quantification of incorporation of full-length bodipy-TMR labeled PIP2 in plasma membrane, and FIG. 7C shows the trafficking of short chain Top fluor-bodipy labeled PIP2 to endosomes and Golgi over time in unloaded cells. The short chain analogue is modified to prevent metabolization of PIP2 inside the cells (ester bond replaced by ether bond). The Bodipy-TMR-PIP2 will allow us to determine trafficking of PIP2 directly from PM, while Bodipy-PIP2 will allow us to determine trafficking of PIP2 from ER-Golgi to nucleus.

Cholesterol loading is expected to either alter the expression or localization of TMEM55b (decreased expression and lower nuclear localization) or PIP5Kα (increased expression and increased nuclear localization), while cholesterol depletion is expected to have opposite effects; TMEM55b (increased expression and higher nuclear localization) and PIP5Kα (decreased expression and reduced nuclear localization). There is a possibility that increased or reduced levels of PIP2 metabolic enzymes in nucleus are due to shutlling between cytoplasm and nucleus. As an alternative strategy, we can use nuclear export and import blockers. To block nuclear export, cholesterol-loaded or depleted cells will be treated with low dose of leptomycin B (LMB). To block nuclear import, cells will be treated with ivermectin. Blocking nuclear export in cholesterol-loaded cells is expected to increase nuclear TMEM55b, while blocking nuclear import is expected to reduce nuclear PIP5Kα. We can also use other inhibitors such as importazoole (for nuclear import) or CBS9106 (for nuclear export).

Nuclear PIP2 regulates ABCA1 expression via direct binding to RXR. Applicants observed reduced ABCA1 mRNA levels in PIP2 depleted cells (FIG. 3D), thus PIP2 may play a role in either stabilizing ABCA1 mRNA or in increasing transcription of ABCA1. PIP2 levels in nucleus increases upon cholesterol loading and we identified a novel PIP2 binding domain in RXR, located between DNA binding domain and retinoic acid binding domain. Thus, PIP2 may be modulating ABCA1 levels via direct interaction with RXR.

Applicants will employ two cell lines THP-1 and HepG2 (These cell lines express ABCA1 at basal level and expression can be induced bt LXR agonists) to test the effect of PIP2 levels on ABCA1 transcription. To deplete PIP2 levels, we will use three independent strategies; 1) overexpression of PIP2 phosphatase TMEM55b, 2) ISA2011b treatment, and 3) using Crispr-Cas9 generated PIP5Kα KO cell lines. To increase PIP2 levels, we will use three independent strategies; 1) exogenous delivery of PIP2-liposomes, 2) Crispr-Cas9 generated TMEM55b^{-/-} cell lines and 3) rAAV8 mediated overexpression of PIP2 biosynthetic enzyme PIP5kα. Pilot data with Crispr-Cas9 generated HepG2-TMEM55b^{-/-} cells showed ˜32% increase in PIP2 levels (not shown), indicating feasibility of our approach. We will determine ABCA1 mRNA transcription (nuclear run-off assay) and mRNA turnover assay (by quantifying the mRNA degradation rate using RT-PCR at various times after addition of actinomycin D). To determine if the role of PIP2 in ABCA1 transcription is via LXR/RXR pathway, we will test the effect of PIP2 supplementation on ABCA1 transcription ±T0901317 (LXR-agonist) or GSK2033 (LXR antagonist). Excitingly, our pilot data in THP-1 cells show marked upregulation in ABCA1 mRNA levels upon supplementation with PIP2 liposomes (FIG. 7), indicating feasibility of proposed studies.

To test binding of PIP2 with RXRα, we will use WT-RXR protein (˜117 AA, 50AA on N-terminal side of PIP2 binding domain and 55 AA on C-terminal side, with size of ˜13 KDa) or PIP2 domain mutant isoform of RXR (where K, R and R will be replaced by alanine).

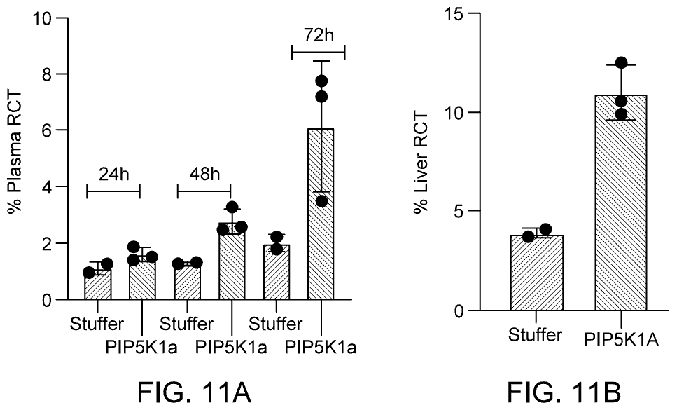

Microscale Thermophoresis (MST): DOPC±PIP2 (mole ratio 1:1) liposomes will be diluted to 16 different concentrations ranging from 30 nM to 1 mM, and 1 μM Alexa 488-labeled RXR (WT or mutant) will be added to all of them. Samples will be loaded into 16 Monolith NT.115 Premium Treated Capillaries (NanoTemper Technologies) and MST will be measured at 25° C. Data from three independent measurements will be analyzed using the signal from the MST-off time of −1.0-0.0 s and MST-on time of 1.5-2.5 s (MO.Affinity Analysis software version 2.2.7, NanoTemper Technologies). Our pilot data showed dose-dependent altered mobility of WT RXR protein with PIP2 containing liposomes, while no difference was found with control DOPC liposomes (FIG. 9A, 9B).

To visualize PIP2-RXR interaction in a micrometer scale, we use cutting-edge STED microscopy and GUVs (±PIP2) doped with 0.1% mole rhodamine-568 labeled PE. The RXR will be labeled with NHS-694 dye at N-terminus and STED microscopy will be used to create super-resolution images by the selective deactivation of fluorophores. Pilot data with WT-RXR showed clear interaction with PIP2 containing GUVs (FIG. 9C).

Liposome flotation and Surface Plasmon resonance (SPR) assays. Briefly, liposomes POPC (control) or POPC-PIP2 and rhodamine labeled protein WT-RXR (control), or RXR-mutant will be placed at the bottom of sucrose density gradient. The samples will be subjected to ultracentrifugation. Samples from bottom, middle and top fraction will be collected and visualized using high-resolution STED microscopy. SPR assays will be performed using SPR2000 instrument. We will immobilize WT or mutant isoform RXR on CMV chip (this chip allows binding of lysine carrying proteins to the surface) and varying amounts of PIP2 will be allowed to flow over the coated surface. In an opposite strategy, biotin labeled PIP2 will be coated on streptavidin chip and varying concentrations of RXR (WT or PIP2 domain mutant) will be allowed to flow over the coated surface. We will employ multiple controls: PE, PC, PS and PI will be used as lipid controls, while LXR will be used a control nuclear protein.

While not wishing to be bound by any theory, Applicants expect PIP2 depletion to increase ABCA1 mRNA turnover or reduce transcription of mRNA. I expect PIP2 supplementation to increase ABCA1 mRNA levels and show further increase in presence of LXR agonist. PIP2 supplementation is not expected to effect mRNA stability. In case we do find increase in mRNA stability, we will conclude that in addition to increasing transcription, PIP2 supplementation also increase mRNA stability of ABCA1. Alternatively, we can also assess the translational activity of ABCA1 mRNA by performing polysome profiling analysis. As an another alternative strategy, we can determine half-life of ABCA1 protein in PIP2 depleted and PIP2 supplemented conditions. As an alternative to TMEM55b, we can test it's close homologue TMEM55a, and other PIP2 phosphatases such as OCRL1 (breaks down PIP2 to PI4p) and synaptojanin (breaks down PIP2 to PI5P). TMEM55b hydrolyzes PI4,5-P2 to PI5P, thus the effects of TMEM55b overexpression on ABCA1 may be mediated by accumulating PI5P rather than depleting levels of PIP2. Applicants can determine the effect of PIP2 depletion on the abundance of candidate microRNAs (miRNAs) such as miR33a, that are known to play role in lipid metabolism and cholesterol efflux. We expect the WT RXR, but not the PIP2 domain-mutant RXR, to show direct binding to PIP2 with expected Kd in nM-μM range. PE, PC, PS and PI are not expected to show any binding with RXR. LXR is not expected to show any binding with PIP2. RXR is not expected to bind other PIP species such as PI3P, PI4P, or PI5P. Alternatively, we can perform Co-IPs (using PIP2 antibody and flag-tagged RXR) from nuclear extracts of cholesterol loaded or depleted cells. As another alternative method, we can use supported bilayer as a lipid template and use varying lipid compositions doped with RhodPE on the coverslip and Alexa 488 labeled RXR. Supported bilayer can be synthesized by bursting the liposomes on charged surface of coverslip bottom chambered slide, followed by injection of 1 μM of Alexa488 RXR to the bilayer solution and using confocal laser microscope to visualize green signal of protein on red-colored lipid bilayer to confirm binding.

PIP2 supplementation increases RCT and reduces atherosclerosis in mice. PIP2 mediated enhancement of ABCA1 expression induces RCT and reduces atherosclerosis.

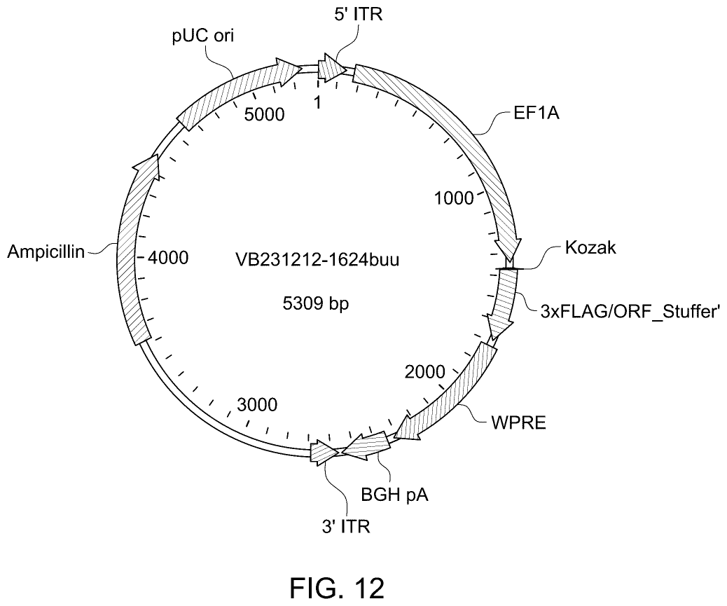

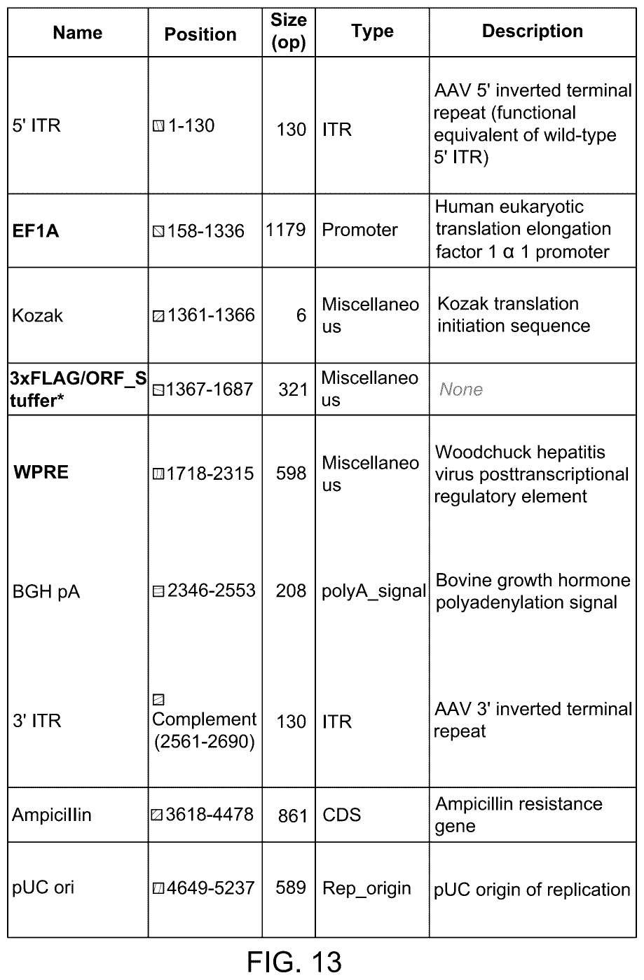

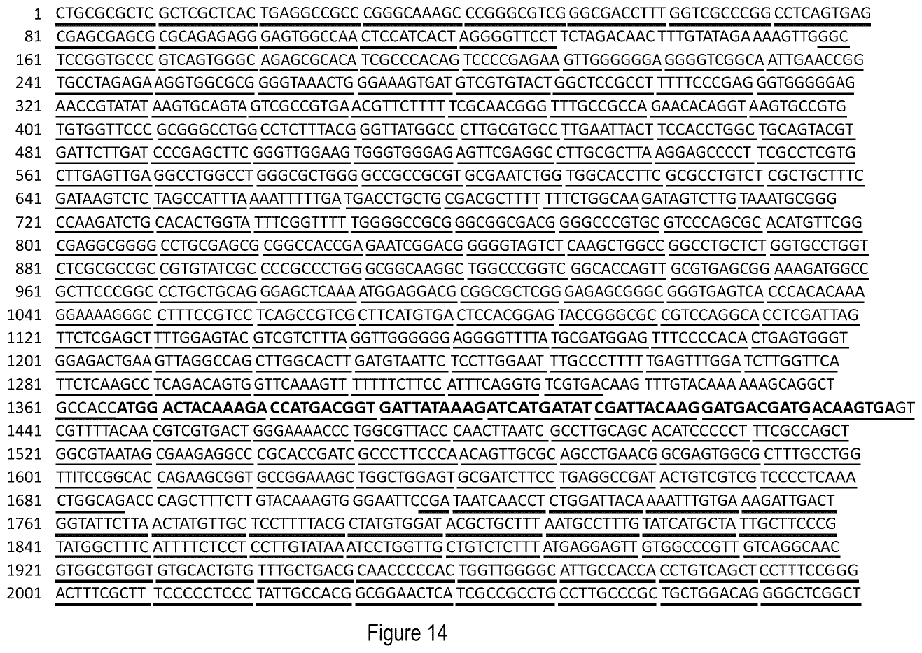

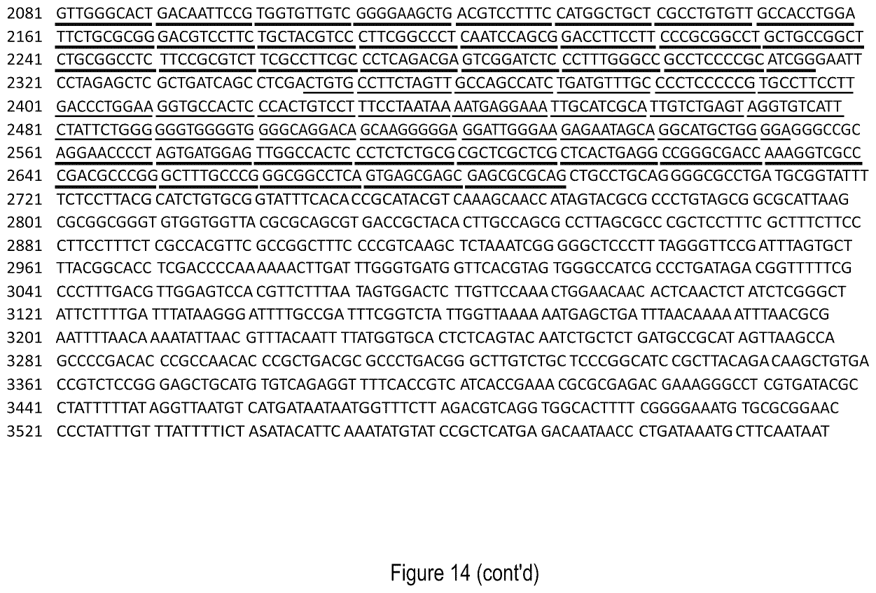

To confirm if the in-vitro effects of PIP2 on ABCA1 expression translate into in-vivo results, we tested the effects of rAAV8-PIP5K1a on ABCA1 levels in liver and intestinal tissue. Both tissues play a significant role in ABCA1-mediated cholesterol efflux from the body. We designed recombinant adeno-associated virus 8 (rAAV8) constructs expressing flag-tagged PIP5K1a under a human eukaryotic translation elongation factor 1 α (EF1) promoter. The CMV promoter was not used as several studies have reported issues in the liver expression of transgenes from CMV promoter. A single dose of empty or PIP5ka-3XFlag vector with a viral titer of 1×10¹³ genome copies (GC)/kg) of viral particles was injected into WT mice via tail-vein injections. The animals were sacrificed post 5 weeks of rAAV8-PIP5K1a injections and protein extracts from the liver and intestinal tissue were prepared. PIP2 levels in plasma, liver, and intestinal tissue were increased by ˜36-45%. Western blot analysis showed expression of flag-tagged PIP5K1a in liver tissue of rAAV8-PIP5K1 injected vs. mice injected with empty vector (FIG. 10A).

Excitingly, the ABCA1 levels in the liver were ˜2-fold higher in male and ˜3-fold higher in female mice. Similarly, the intestinal tissue (ileum) of mice injected with rAAV8-PIP5K1a showed ˜3-fold induction of ABCA1 in both female and male mice vs. control mice (FIG. 10B). To ensure that PIP5Ka supplementation works via the same mechanism, we performed qRT-PCT analysis on liver and Ileum tissue. As shown in FIGS. 10C, and 10D, mice injected with rAAV8-PIP5K1a showed significantly higher mRNA levels of ABCA1. These data provide strong proof of concept for rAAV8-PIP5Ka-mediated induction of ABCA1 levels in animals. The pilot data also showed no reduction in LDLr expression in rAAV8-PIP5K-injected mice vs. control mice. Mechanistically, like in-vitro studies, in-vivo expression of PIP5K1a increased mRNA levels of ABCA1 in the liver and intestine (Ileum).

To further probe the physiological relevance of PIP2 in apoA1-ABCA1 mediated reverse cholesterol transport pathway, WT C57BL6/J mice were either injected with stuffer or rAAV8-PIP5K1a (as described above). After 6 weeks post rAAV8-PIP5K1a injection, radiolabeled foam cells (bone marrow-derived macrophages loaded with Acetylated LDL+³H-cholesterol) were transplanted s.c on the upper-back of mice.

WT C57BL6J mice were euthanized by CO₂ inhalation and femoral bones were removed. The marrow was flushed out of the bones into a 50 mL sterile tube using a 10 mL syringe with a 26-gauge needle filled with sterile DMEM. Cells were centrifuged for 5 min at 1,800 rpm at 4° C., followed by two washes with sterile PBS. The cells were cultured for 11 to 14 days in DMEM supplemented with 20% L-cell conditioned medium, 10% fetal bovine serum and 1% penicillin/streptomycin. To generate foam cells, the tritium labeled ³H-cholesterol (2 μCi/mL; Perkin Elmer, Norwalk, USA) and 100 μg/mL acetylated LDL were mixed and incubated at 37° C. This cholesterol mixture was combined with DMEM containing 20% L-cell conditioned medium and BMDMs were incubated with cholesterol-labeled media for 48 h to generate foam cells. The cholesterol loaded foam cells were washed twice with DMEM prior to harvesting for in vivo injection, and ˜2 million ³H-cholesterol dpm in a volume of 0.25 mL were transplanted subcutaneously on the upper back of recipient mice. At 24, 48, and 72 h post transplantation, plasma samples were collected. Plasma radioactivity was determined, and total plasma dpm was calculated by estimating blood volume to be equal to 7% of the body weight and plasma to be 55% of the blood volume. RCT to the plasma was calculated as the % (dpm appearing in plasma/total dpm injected) of injected radioactivity. Upon sacrifice at 72 h, the liver was removed and weighed. A piece of liver, ˜0.2 g, was isolated, weighed, suspended in PBS, homogenized, and a known amount of ¹⁴C-cholesterol radioactivity was added as a recovery standard. The radioactivity in an aliquot of 0.3 mL of the liver homogenate was measured by liquid scintillation counting. The ¹⁴C-cholesterol dpm was used to back calculate the [³H] recovery for the entire liver homogenate, which was further used to calculate the total amount of radioactivity in the liver. In addition to ¹⁴C-cholesterol internal standard, all RCT data was standardized to mouse body each harvest time point. As shown in pilot data from male mice (N=3), the RCT to plasma was increased at 48 h and 72 h in mice injected with rAAV8-PIP5K1a vs. control mice. Similar trend was found in liver RCT, with mice injected with rAAV8-PIP5K1a showing higher RCT to liver vs. control mice.

Applicants further expect C57BL6/J mice injected with PIP2 liposomes to show similar increases in ABCA1 expression in liver tissue. ABCG1 (other protein involved in RCT) and plasma apoA1 levels are an alternative endpoint. RCT to plasma, liver, and feces will be higher in mice treated with PIP2 liposomes vs. mice injected with control liposomes. PIP2 supplemented mice are expected to show reduced total plasma cholesterol levels. PIP2 supplemented apoE^{-/-} KO mice fed with WTD are expected to show at least one of reduced atherosclerotic plaque area, reduced macrophage infiltration in plaque area, and reduced collagen deposition vs. mice injected with control liposomes. The PIP2 liposome stability and circulation time may also be increased via PEGylation of PIP2 liposomes. As another alternative method, we test the effect of PIP2 supplementation on regression of existing atherosclerotic plaques. In certain embodiments, the mice will be divided into two groups wherein 1) injected with control POPC liposomes and 2) injected with POPC-PIP2 liposomes (20 mg/kg).

What has been described above includes examples of one or more embodiments. It is, of course, not possible to describe every conceivable modification and alteration of the above compositions or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

1. A method of treating, preventing, inhibiting, or ameliorating the symptoms of a disease or disorder that is modulated or otherwise affected by ABCA1 levels and cholesterol efflux or in which ABCA1 levels and cholesterol efflux is implicated, the method comprising administering to an individual in need thereof a therapeutically effective amount of PIP2 or an expression vector capable of inducing expression of PIP2 in the individual.

2. The method of claim 1, wherein the expression vector has 80% to 99% sequence identity to rAAV8-PIP5K1a.

3. The method of claim 1, wherein the PIP2 is provided in a delivery vehicle, including but not limited to a liposome-based vehicle.

4. The method of claim 1, wherein the disease or disorder is hypercholesterolemia, hyperlipoproteinemia, hypertriglyceridemia, lipodystrophy, hyperglycemia, diabetes mellitus, dyslipidemia, atherosclerosis, Parkinson's disease, Alzheimer's disease, inflammation, immunological disorders, lipid disorders, obesity, efferocytosis, or cardiovascular disorder.

5. The method of claim 1, wherein the individual in need thereof is a human adult.

6. A method of increasing PIP2 level to treat a disease or disorder that is modulated or otherwise affected by ABCA1 levels and cholesterol efflux or in which ABCA1 levels and cholesterol efflux is implicated, the method comprising administering to a subject in need thereof a therapeutically effective amount of PIP2 or an expression vector capable of inducing expression of PIP2 in the individual.

7. The method of claim 6, wherein the expression vector has 80% to 99% sequence identity to rAAV8-PIP5K1a.

8. The method of claim 6, wherein the PIP2 is provided in a delivery vehicle, including but not limited to a liposome.

9. The method of claim 6, wherein the disease or disorder is hypercholesterolemia, hyperlipoproteinemia, hypertriglyceridemia, lipodystrophy, hyperglycemia, diabetes mellitus, dyslipidemia, atherosclerosis, Parkinson's disease, Alzheimer's disease, inflammation, immunological disorders, lipid disorders, obesity, efferocytosis, or cardiovascular disorder.

10. The method of claim 6, wherein the individual in need thereof is a human adult.

11. The method of claim 6, wherein the disease or disorder is dyslipidemia, wherein the administration leads to a reduction in blood cholesterol concentration of the individual.

12. The method of claim 6, wherein the disease or disorder is cholesterol-induced atherosclerosis, wherein the administration leads to a reduction in blood cholesterol concentration of the individual.

13. The method of claim 6, wherein the administration disease or disorder is efferocytosis.

14. A method of increasing cholesterol efflux from cells of a subject, comprising administering an effective cholesterol efflux-increasing amount of PIP2 or an expression vector capable of inducing expression of PIP2 to an individual in need thereof, wherein the administration increases a level of cholesterol efflux.

15. The method of claim 14, wherein the administering increases a level of ABCA1 expression.


SEQUENCE LISTING (8 sequences)

SEQ ID NO: 1 - protein, 12 aa, synthetic construct
  VARIANT 2..9: note = X can be any amino acid
KXXXXXXXXKKR

SEQ ID NO: 2 - protein, 11 aa, synthetic construct
KVKSSSWRRER

SEQ ID NO: 3 - protein, 10 aa, synthetic construct
KRKGLFARRR

SEQ ID NO: 4 - protein, 12 aa, synthetic construct
KKKTSPLNFKKR

SEQ ID NO: 5 - protein, 12 aa, synthetic construct
KKGGGSSTLSRR

SEQ ID NO: 6 - protein, 12 aa, synthetic construct
KYDRKGYKPRPR

SEQ ID NO: 7 - protein, 12 aa, synthetic construct
KDCLIDKRQRNR

SEQ ID NO: 8 - DNA, 5309 bp, synthetic construct
  source 1..5309: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggactacaaagaccatgacggtgattataaagatcatgatatcgattacaaggatgacgatgacaagtgagtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgagtggcgctttgcctggtttccggcaccagaagcggtgccggaaagctggctggagtgcgatcttcctgaggccgatactgtcgtcgtcccctcaaactggcagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag